(19) United States
(12) Patent Application Publication
(10) Pub. No.: US 2020/0308548 A1
(43) Pub. Date: Oct. 1, 2020

(54) Amplifying Beta Cell Differentiation with Small Molecules BET (Bromodomain And Extraterminal Family Of Bromodomain-Containing Proteins) Inhibitors

(71) Applicants: Centre National de la Recherche Scientifique (CNRS), Paris (FR); lnstitut National de Ia Sante et de Ia Recherche Medicale (INSERM), Paris (FR); Universite Paris Descartes, Paris (FR); Novo Nordisk A/S, Bagsværd (DK)

(72) Inventors: Lukas Huijbregts, Paris (FR); Raphael Scharfmann, Paris (FR); Christian Honore, Copenhagen (DK)

(21) Appl. No.: 16/313,266

(22) PCT Filed: Jun. 30, 2017
(86) PCT No.: PCT/EP2017/066243

§ 371 Date: Dec. 26, 2018

(30) Foreign Application Priority Data
Jul. 1, 2016 (EP) 16305835.7

(51) Int. Cl. C12N 5/071; A61K 35/39; C07K 14/62; A61K 31/4745

(52) U.S. Cl. CPC C12N 5/0676; A61K 35/39; C07K 14/62; A61K 31/4745; C12N 2506/45; C12N 2500/38; C12N 2533/00; C12N 2501/999; C12N 2501/16; C12N 2501/998; C12N 2501/117; C12N 2506/02

(57) ABSTRACT

The present invention provides an in vitromethod for obtaining cells of the pancreatic endocrine lineage, comprising a step of culturing pancreatic progenitor cells, wherein said pancreatic progenitor cells are in a cell culture medium comprising at least one BET inhibitor.

The present invention provides a method for obtaining cells of the pancreatic endocrine lineage, comprising a step of culturing pancreatic progenitor cells with at least one BET inhibitor.

The endocrine pancreas plays a crucial role in nutritional homeostasis through synthesis and secretion of hormones by cells aggregated into islets of Langerhans. The latter contain four different cell subtypes: α-, ß-, δ-, and PP-cells, which produce glucagon, insulin, somatostatin, and Pancreatic Polypeptide, respectively. Insulin and glucagon function coordinately to control glucose homeostasis, insulin preventing hyperglycemia, glucagon exerting the opposite activity (Pan and Wright, 2011).

Diabetes is characterized by high blood glucose levels, which, in most cases, result from the inability of the pancreas to secrete sufficient amounts of insulin. While type 1 diabetes (T1D) is caused by the autoimmune-mediated destruction of insulin-producing ß-cells, type 2 diabetes (T2D) results from ß-cell failure and eventual loss over time. Current treatments of diabetes fail to strictly restore normoglycemia. Therefore, replenishing the pancreas with new functioning ß-cells and/or maintaining the health of the remaining ß-cells represent key strategies for the treatment of both conditions. In this context, deciphering the mechanisms underlying ß-cell genesis and/or regeneration may uncover new avenues towards alternative therapies based on drug discovery, cell therapy, or regenerative medicine.

ß-cells develop through a tightly regulated multistep process. During prenatal life, pancreatic progenitors proliferate and, subsequently, differentiate into endocrine progenitors that give rise to endocrine cells, including insulin-producing ß-cells. During early postnatal life, such ß-cells proliferate further prior to acquiring functional properties, such as glucose-regulated insulin secretion. Later on, ß-cells proliferate at a much lower rate (Jennings et al., 2015). Interestingly, recent data indicate that, in adult rodent pancreas, newly formed ß-cells can also arise by conversion of non-ß-cells into ß-cells (Avolio et al., 2013).

During the past decades, using transgenic mice, a set of transcription factors was found to play a major role at specific steps of the ß-cell development. Signals necessary to promote pancreatic progenitor cell proliferation and their differentiation into ß-cell were described by (Gittes, 2009). Such information was therefore used for the establishment of in vitro protocols aiming at generating ß-cells from human embryonic stems cells (hESC) or induced pluripotent stem cells (iPSC). An important first breakthrough appeared in 2006 when the biotech company NovoCell, now known as Viacyte, published a protocol to generate pancreatic hormone-expressing endocrine cells from hESC in vitro (D'Amour et al., 2006). This protocol attempted to mimic pancreatic development in a step-wise fashion, hESC being successively differentiated into definitive endoderm, gut-tube endoderm, pancreatic progenitors, endocrine progenitors, and finally hormone-producing endocrine cells. The protocol appeared quite robust but insulin-producing cells were not similar to genuine ß-cells as they co-expressed additional hormones in addition to insulin, such as glucagon, and secreted insulin in a glucose-independent manner. This protocol, however, represented the basis for a second study performed by the same group. There, the authors demonstrated that, when grown in the right environmental setting (transplantation into immune-deficient SCID mice, used here as an incubator for cell differentiation), pancreatic progenitors derived from hESC gave rise to functional ß-cells (Kroon et al., 2008). This work represented a breakthrough demonstrating that pancreatic progenitors can be generated from hESC, providing the proper environment (in this case, an immune-incompetent mouse). More recently, additional progress was made towards the in vitro generation of functional human ß-cells from hESC. Different teams implemented protocols based on the aforementioned NovoCell/Viacyte one (Pagliuca et al., 2014; Rezania et al., 2014a). Such modified protocols gave rise to mono-hormonal insulin-containing cells, whose phenotype, based on extensive series of marker analyses, resembled primary human ß-cells. The cells produced large amounts of insulin as seen in human ß-cells. However, the efficiency of the differentiation process remained limited and the induction of insulin secretion by glucose appeared relatively weak when compared to genuine human islet preparations (Rezania et al., 2014a). Moreover, information on the efficiency of the process remains scarce. In conclusion, during the last 15 years, progresses were made towards the establishment of protocols allowing the differentiation of hESC into functional human ß-cells. However, additional efforts remain required to discover molecules and pathways that could potentiate the development of functional ß-cells from pancreatic progenitors.

Previous assay to screen for signals that modulate pancreatic progenitor cell proliferation identified the major role of FGFR2IIIb ligands in this process. Specifically, Pdx1+ pancreatic progenitors express FGFR2IIIb that interacts with FGF7 and FGF10, two key factors produced by the surrounding mesenchyme. Both FGF7 and FGF10 induce the amplification of pancreatic progenitors in rodents (Bhushan et al., 2001; Elghazi et al., 2002; Miralles et al., 1999). Importantly, these findings were confirmed in the human fetal pancreas (Ye et al., 2005). Therefore, FGF7 and FGF10 are now used in nearly all protocols aiming at amplifying pancreatic progenitor cells from hESC (Chen et al., 2009; D'Amour et al., 2006; Kroon et al., 2008; Pagliuca et al., 2014; Rezania et al., 2014a).

Therefore, there still remains a significant need to develop methods for differentiating pluripotent stem cells into cells of the pancreatic endocrine lineage, pancreatic hormone expressing cells, or pancreatic hormone secreting cells.

In this context, the applicant unexpectedly found that BET (bromodomain and extraterminal family of bromodomain-containing proteins) inhibitors could be used to differentiate immature embryonic pancreases by mimicking the classical steps of pancreas development.

Therefore, the present invention provides an in vitro method for obtaining cells of the pancreatic endocrine lineage, comprising a step of culturing pancreatic progenitor cells, wherein said pancreatic progenitor cells are in a cell culture medium comprising at least one BET inhibitor, and wherein said pancreatic progenitor cells are obtained by differentiation of stem cells obtained by techniques that do not involve the destruction of a human embryo.

A BET inhibitor inhibits the binding of BET family bromodomains to acetylated lysine residues. By “BET family bromodomains” it is meant a polypeptide comprising two bromodomains and an extraterminal (ET) domain or a fragment thereof having transcriptional regulatory activity or acetylated lysine binding activity. Exemplary BET family members include BRD2, BRD3, BRD4 and BRDT are given in WO 2011/143669. Examples of BET inhibitors include but are not limited to the compounds of the instant invention. Advantageously, the BET inhibitor according to the invention is targeting BD1 and/or BD2, and is preferentially selected in the group comprising BET151, JQ1, BET762, OXT-015, TEN-010, CPI-203, CPI 0610,l LY29002 and RVX8, preferentially BET 151 and JQ1.

In a preferred embodiment, the cell culture medium according to the invention, comprised said at least BET inhibitor in a concentration from 10 nM to 10 μM, preferentially from 0.1 μM to 1 μM, from 0.3 μM to 0.8 μM or from 0.4 μM to 0.6 μM.

In a preferred embodiment, pancreatic progenitor cells are cultured in a cell culture medium according to the invention from 8 hours to 96 hours, preferably from 24 hours to 72 hours, more preferably from 48 hours to 72 hours.

In a preferred embodiment, pancreatic progenitor cells are cultured in a cell culture medium according to the invention for at least 8 hours, preferably for at least 24 hours, more preferably for 48 hours, even more preferably 72 hours.

As used herein, a “cells of the pancreatic endocrine lineage” refers to pancreatic endocrine islet cells or progenitor thereof. Cells of the pancreatic endocrine lineage are characterized as cell with positive gene expression for the transcription factor Pdx-1 and at least one of the following transcription factors: NGN3, NKX2.2, NKX6-1, NEUROD, ISL-1, HNF3 beta, MAFA, PAX4, ARX or PAX6. Pax4 and ARX specify the ß/δ- and α-cell destinies, respectively (Avolio et al., 2013).Cells expressing markers characteristic of the pancreatic cell lineage refers to a cell capable of expressing at least one of the following hormones: insulin, glucagon, somatostatin, or pancreatic polypeptide. Preferably, according to the invention, a cell will be considered as a precursor of endocrine islets cells if it expresses at least the marker NGN3.

As used herein, a “pancreatic progenitor cells” refer to an undifferentiated pancreatic cell, initially expressing specific transcription factors, such as PDX1, that are committed to a specific developmental pathway to differentiate into functional pancreatic endocrine cells capable of expressing at least one of the following hormones: insulin, glucagon, somatostatin, or pancreatic polypeptide. As used herein, a “differentiation” refers to a process where cells progress from an undifferentiated state to a differentiated state or from an immature state to a mature state. For example, undifferentiated pancreatic cells are able to proliferate and express characteristics markers, like PDX-1. Mature or differentiated pancreatic cells do not proliferate and secrete high levels of pancreatic endocrine hormones. Changes in cell interaction and maturation occur as cells lose markers of undifferentiated cells or gain markers of differentiated cells. Loss or gain of a single marker can indicate that a cell has “matured or differentiated”.

By “cell culture medium” it is meant any culture medium know in the art suitable for culturing pancreatic progenitor cells, which are characterized in particular by the expression of the markers PDX1⁺/NKX6-1⁺/NEUROD1⁺ Rezania et al. (2014). Such culturing media are for example disclosed in Rezania et al. (2014). By “Markers” it refers to nucleic acid or polypeptide molecules that are differentially expressed in a cell of interest, increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

Preferably, the cell of the pancreatic endocrine lineage obtainable by the method of the invention is a functional beta cell, that is to say a cell displaying a functional beta cell phenotype.

In the context of the invention, the terms “beta cell activity” refer to the production of insulin and/or C-peptide by cell of the pancreatic endocrine lineage upon glucose stimulation, which can easily be assayed by techniques well known from the person skilled in the art, such as ELISA. Such techniques have for example been described in Baeyens et al. (Diabetologia; 48(1):49-57; 2005). Preferably, the terms “beta cell activity” refer to the production of insulin by differentiated beta cell.

According to the invention, the terms “comprising” or “containing” mean, without limitation, the inclusion of the referent and do not exclude the presence of other elements. For example, “a method comprising the step of x” encompasses any method in which x is carried out, independently of the fact that additional steps are also performed. Likewise, “a composition comprising x and y” encompasses any composition that contains x and y, no matter what other components may be present in the composition.

In contrast, the terms “consisting of” mean the inclusion of the referent and the exclusion of any element not explicitly listed. When referring to a method, the terms “consisting of the steps x, y, and z” encompass methods in which steps x, y and z are performed, and wherein non-listed steps are not.

Preferentially, the pancreatic progenitor cells according to the invention are obtained from embryonic stem cells, perinatal stem cell, somatic stem cells, and bioengineered stem cells, preferably said stem cells are hESC, or iPSC, in particular hiPSC.

In an embodiment, when stems cells are human stem cells, said human stem cells are not human embryonic stem cells.

In the context of the invention, the stem cells can be any of the stem cells as defined below. According to the invention, the terms “mammalian stem cells” encompass mammalian embryonic stem cells, mammalian perinatal stem cell, mammalian somatic stem cells, and mammalian bioengineered stem cells. Preferably, according to the invention, mammalian stem cells are chosen from the list consisting in mammalian embryonic stem cells, mammalian perinatal stem cell, mammalian somatic stem cells, and mammalian bioengineered stem cells.

By “mammalian embryonic stem cells”, it is herein referred to mammalian stem cells derived from the inner cell mass (ICM) of a mammalian embryo at the blastocyst stage.

By “embryo” it is herein referred to a multicellular diploid eukaryote in its earliest stage of development, from the time of first cell division until birth, hatching, or germination.

By “blastocyst” it is herein referred to a structure formed in the early development of mammals. Typically, it possesses an inner cell mass (ICM), or embryoblast, which subsequently forms the embryo, and an outer layer of cells, or trophoblast, surrounding the inner cell mass and a fluid-filled cavity known as the blastocoele.

According to the invention, mammalian embryonic stem cells may be either obtained from an established cell line, or isolated from an embryo by different techniques.

Techniques for isolating a stem cell from an embryo are well known from the person skilled in the art, and include either technique that involve the destruction of an embryo when the embryo is not a human embryo, or techniques that do not involve the destruction of an embryo.

Briefly, conventional techniques for obtaining embryonic stem cells (which involve the destruction of an embryo) may comprise the steps of isolating a primate blastocyst, isolating cells from the inner cellular mass (ICM) of the blastocyst, plating the ICM cells on a fibroblast layer (wherein ICM-derived cell masses are formed) removing an ICM-derived cell mass and dissociating the mass into dissociated cells, replating the dissociated cells on embryonic feeder cells and selecting colonies with compact morphology containing cells with a high nucleus/cytoplasm ratio, and prominent nucleoli. The cells of the selected colonies are then cultured.

For example, conventional techniques for obtaining embryonic stem cells (which involve the destruction of an embryo) have been described in U.S. Pat. No. 5,843,780, and by Thomson et al. (Science; 282: 1145-1147; 1998; Curr. Top. Dev. Biol; 38: 133; 1998; Proc. Natl. Acad. Sci. U.S.A., 92:7844; 1995).

On the other hand, embryonic stem cells can be extracted from human embryos without resulting in embryo destruction, using a technique used in pre-implantation genetic diagnosis.

For example, a technique that does not involve the destruction of an embryo has been described by Chung et al. (Cell Stem Cell; 2(2):113-7; 2008).

In an embodiment, mammalian stem cells are human stem cells.

By “human embryonic stem cells” or “hESC, it is herein referred to human stem cells derived from the inner cell mass (ICM) of a human embryo at the blastocyst stage. Human embryos reach the blastocyst stage 4-5 days post fertilization, at which time they consist of between 50 and 150 cells. Embryonic stem cells are pluripotent stem cells.

According to the invention, human embryonic stem cells may be either obtained from an established cell line, or isolated from an embryo by different techniques known from the person skilled in the art.

By “human embryo” it is herein referred to a multicellular diploid eukaryote in its earliest stage of development, from the time of first cell division until about eight weeks after fertilization (or about ten weeks after the last menstrual period). By contrast, a multicellular diploid eukaryote after more than about eight weeks after fertilization and before birth is called a fetus.

Non-limiting examples of human embryonic stem cells lines are for example the cell lines CHB-1, CHB-2, CHB-3, CHB-4, CHB-5, CHB-6, CHB-8, CHB-9, CHB-10, CHB-11, CHB-12, Rockefeller University Embryonic Stem Cell Line 1 (RUES1), Rockefeller University Embryonic Stem Cell Line 2 (RUES2), HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 26, HUES 27, HUES 28, CyT49, Rockefeller University Embryonic Stem Cell Line 3 (RUES3), WA01 (H1), UCSF4, NYUES1, NYUES2, NYUES3, NYUES4, NYUES5, NYUES6, NYUES7, HUES 48, HUES 49, HUES 53, HUES 65, HUES 66, UCLA 1, UCLA 2, UCLA 3, WA07 (H7), WA09 (H9), WA13 (H13), WA14 (H14), HUES 62, HUES 63, HUES 64, CT1, CT2, CT3, CT4, MA135, Endeavour-2, WIBR1, WIBR2, HUES 45, Shef 3, Shef 6, WIBR3, WIBR4, WIBR5, WIBR6, BJNhem19, BJNhem20, SA001, SA002, UCLA 4, UCLA 5, UCLA 6, HUES, ESI-014, ESI-017, WA15, WA17, WA18, WA19, WA20, WA21, WA22, WA23, WA24, CSES2, CSES4, CSES7, CSES8, CSES11, CSES12, CSES13, CSES14, CSES15, CSES17, CSES19, CSES20, CSES21, CSES22, CSES23, CSES24, CSES25, HAD-C 100, HAD-C 102, HAD-C 106, ESI-035, ESI-049, ESI-051, ESI-053, CSES5, CSES6, CSES18, CA1, CA2, MEL-1, MEL-2, MEL-3, MEL-4, UCLA 8, UCLA 9, UCLA 10, UM4-6, GENEA002, GENEA048, Elf1, HUES 42, HUES 44, NMR-1, UM14-1, UM14-2, HUES 68, HUES 70, HUES 69, HUES PGD 10, UCLA 11, UCLA 12, WA25, WA26, WA27, H5346, H5401, HS420, I3 (TE03), 14 (TE04), 16 (TE06), UM22-2, CR-4, KCL011, GENEA015, GENEA016, GENEA047, GENEA042, GENEA043, GENEA057, GENEA052, SA121.

For ethical reasons, the present invention preferably does not pertain to objects that may be considered as contrary to “ordre public” or morality. Therefore, in the context of the invention, the terms “human embryonic stem cells” preferably refer to human embryonic stem cells which isolation has not involved the destruction of an embryo. In other words, the terms “human embryonic stem cells” preferably exclude human embryonic stem cells isolated by techniques that involve the destruction of an embryo.

In the context of the invention, it is to be understood that any technique that does not involve the destruction of an embryo can be used, including those that are not described herein.

Moreover, in the context of the invention, the embryos used for obtaining human embryonic stem cells are preferably embryos that cannot give rise to a human being, such as embryos destined to be discarded following in vitro fertilization (IVF) and embryos created solely for the purpose of stem cell research.

Hence, in a yet preferred embodiment, the terms “human embryonic stem cells” preferably refer to human embryonic stem cells isolated from discarded embryos or research embryos, advantageously by techniques that do not involve the destruction of an embryo.

By “discarded embryos” it is herein referred to embryos specifically created in the process of an in vitro fertilization and declared unwanted by the human subjects it originates from.

By “research embryos” it is herein referred to embryos specifically used for scientific research. By “mammalian perinatal stem cells”, it is herein referred to mammalian stem cells derived from the amniotic fluid, placenta, maternal blood supply, umbilical cord and Wharton's Jelly.

By “human perinatal stem cells”, it is herein referred to human stem cells derived from the amniotic fluid, placenta, maternal blood supply, umbilical cord and Wharton's Jelly. Such cells can thus be obtained from tissue samples rather than human embryos, the destruction of which they do not require. Those cells have been thoroughly described in Cetrulo et al. (Perinatal Stem Cells, Second Edition, Wiley-Blackwell, 2013), which the person skilled in the art may refer to.

By “human somatic stem cells” or “human adult stem cells” it is herein referred to stem cells found throughout the human body after birth. Such cells can thus be obtained from adult tissue samples rather than human embryos, the destruction of which they do not require.

According to the invention, “human somatic stem cells” encompass hematopoietic stem cells, mesenchymal stem cells, endothelial stem cells, neural stem cells, olfactory adult stem cells, neural crest stem cells, and testicular cells.

Cells derived from bone marrow and amniotic fluid, which can include both hematopoietic stem cells and mesenchymal stem cells, have been found to differentiate into beta cells with manipulation in an in vitro environment (Jiang et al.; Nature.; 418:41-4; 2002, and De Coppi et al.; Nat Biotechnol.; 25:100-106 ; 2007).

Preferably, the term “human somatic stem cells” refers to hematopoietic stem cells and mesenchymal stem cells.

According to the invention, the term “hematopoietic stem cells” refers herein to a stem cell displaying a hematopoietic stem cells phenotype.

By “hematopoietic stem cells phenotype” it is herein meant the expression of at least one hematopoietic stem cells marker, and/or the presence of hematopoietic stem cells morphology.

Examples of typical hematopoietic stem cells markers include, without limitation, CD34+, CD59+, Thy1/CD90+, CD38lo/−, and C-kit/CD117+.

As regards their morphology, hematopoietic stem cells are non-adherent and rounded cells, with a rounded nucleus and low cytoplasm-to-nucleus ratio. They can further be identified by their small size, lack of lineage (lin) markers, low staining (side population) with vital dyes such as rhodamine 123 (rhodamineDULL, also called rholo) or Hoechst 33342, and presence of various antigenic markers on their surface.

Hematopoietic stem cells can be found in bone marrow and bone marrow biological samples. According to the invention, the term “mesenchymal stem cells” refers herein to a stem cell displaying a mesenchymal stem cell phenotype.

By “mesenchymal stem cell phenotype” it is herein meant the expression of at least one mesenchymal stem cells marker, and/or the presence of a mesenchymal stem cell morphology.

Examples of typical mesenchymal stem cell markers include, without limitation, CD73, CD90 and CD105. Mesenchymal stem cells lack the expression of the markers CD11b, CD14, CD19, CD34, CD45, CD79a and HLA-DR.

As regards their morphology, mesenchymal stem cells are characterized by a small cell body with a few cell processes that are long and thin. The cell body contains a large, round nucleus with a prominent nucleolus, which is surrounded by finely dispersed chromatin particles, giving the nucleus a clear appearance. The remainder of the cell body contains a small amount of Golgi apparatus, rough endoplasmic reticulum, mitochondria, and polyribosomes. The cells, which are long and thin, are widely dispersed and the adjacent extracellular matrix is populated by a few reticular fibrils but is devoid of the other types of collagen fibrils.

Mesenchymal stem cells can be found for example in placenta, adipose tissue, lung, bone marrow and blood, Wharton's jelly from the umbilical cord, muscle, and teeth (perivascular niche of dental pulp and periodontal ligament).

“Stem cell” are undifferentiated cell which has the ability to both self-renew (through mitotic cell division) and undergo differentiation to form a more specialized cell. Stem cells have varying degrees of potency. A precursor cell is but one example of a stem cell. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

By “bioengineered stem cells”, it is herein referred to pluripotent stem cells artificially derived from a non-stem cell. In the context of the invention, the terms “bioengineered stem cells”, encompass pluripotent stem cells obtained from somatic cell nuclear transfer (SCNT, those cells are hereafter referred to as “SCNT cells”) and cells obtained from pluripotency-induced by compounds-mediated reprogramming (those cells are hereafter referred to as induced pluripotent stem cells, iPS cells or iPSCs).

By “human induced pluripotent stem cells”, also abbreviated as iPS cells or iPSCs, it is herein referred to pluripotent stem cells artificially derived from a non-pluripotent cell by compound mediated reprogramming. In the context of the invention, compound-mediated reprogramming includes for instance factor-mediated reprogramming and small-molecule compounds reprogramming.

Briefly, in factor-mediated reprogramming, iPS cells can be derived from somatic stem cells by inducing the expression of a number of specific genes, considered pluripotent related transcription factors.

Another object of the present invention relates to a cell of the pancreatic endocrine lineage obtainable by a method according to the invention.

Still another object of the present invention is a cell of the pancreatic endocrine lineage according to the invention, for use as a medicament.

In a preferred embodiment, the cell of the pancreatic endocrine lineage according to the invention is used as a medicament for treating or preventing a pancreatic disorder, preferably chosen in the list consisting of pancreatitis, such as acute pancreatitis and chronic pancreatitis, diabetes mellitus, exocrine pancreatic insufficiency (EPI), cystic fibrosis (also known as mucoviscidosis), congenital malformations, such as pancreas divisum and annular pancreas, neoplasms (such as serous cystadenoma of the pancreas, solid pseudopapillary neoplasm or Zollinger-Ellison syndrome), and Hemosuccus pancreaticus.

As used herein, “treatment” includes prophylactic and curative intervention in a disease process. Thus, the term “treatment” as used herein, typically refers to therapeutic methods for reducing or eliminating the symptoms of the particular disorder for which treatment is sought. The term “subject” as used herein, generally refers to any warm-blooded mammal, such as humans, non-human primates, rodents, and the like, which is to be the recipient of the particular treatment.

Preferably, the cell of the pancreatic endocrine lineage according to the invention is used as a medicament for treating or preventing diabetes mellitus, more preferably type I or type II diabetes.

The present invention also relates to the use of a cell of the pancreatic endocrine lineage obtainable by a method according to the invention for the in vitro production of insulin.

As used herein, “in vitro production of insulin” refers to insulin producing cells cultured in vitro and capable of secreting detectable amounts of insulin. “Insulin producing cells” can be individual cells or collections of cells. “Insulin producing cells” can be obtained from stem cells or pancreatic from pancreas. One example of a collection of “insulin producing cells” is “insulin producing cell aggregates” e.g., an organized collection of cells with a surrounding mantle of CK-19 positive cells and an inner cell mass.

As used herein, “pancreatic cells from a pancreas” or “a culture of pancreatic cells from a pancreas” refers to a total cell population isolated from a donor pancreas and includes e.g., both pancreatic endocrine cells and pancreatic exocrine cells.

As used herein, “pancreatic exocrine cells” refers to pancreatic cells that secrete pancreatic enzymes for digestion into the gastrointestinal tract. Pancreatic enzymes include e.g., trypsin, chymotrypsin, and carboxypeptidase. Measurement of levels of pancreatic enzyme nucleic acids and proteins can thus be used to determine the presence or absence of pancreatic exocrine cells in a cell population. Pancreatic exocrine cells include cells at all stages of development, e.g., progenitor cells, dividing cells, and mature enzyme secreting cells.

The present invention further relates to the use of a cell of the pancreatic endocrine lineage obtainable by a method according to the invention for the in vitro identification of compounds capable of modulating insulin production.

As employed herein, the term “modulating” refers to the capacity to alter a measurable functional property of biological activity or process (e.g., insulin production).

Another object of the present invention is at least one BET inhibitor for use for treating or preventing a pancreatic disorder, preferably chosen in the list consisting of pancreatitis, such as acute pancreatitis and chronic pancreatitis, diabetes mellitus, exocrine pancreatic insufficiency (EPI), cystic fibrosis (also known as mucoviscidosis), congenital malformations, such as pancreas divisum and annular pancreas, neoplasms (such as serous cystadenoma of the pancreas, solid pseudopapillary neoplasm or Zollinger-Ellison syndrome), and Hemosuccus pancreaticus. Advantageously, the pancreatic disorder according to the invention is diabetes mellitus, preferably type I or type II diabetes.

Another object of the present invention is a pharmaceutical composition comprising at least one BET inhibitor according to the invention and a pharmaceutically acceptable carrier. The term “pharmaceutically acceptable carrier” (or medium) refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Standard pharmaceutical carriers are sterile solutions, tablets, coated tablets, and capsules. Typically, such carriers contain excipients such as starch, milk, sugar, certain types of clay, gelatin, stearic acids or salts thereof, magnesium or calcium stearate, talc, vegetable fats or oils, gums, glycols, or other known excipients. Examples of pharmaceutically acceptable carriers include, but are not limited to, the following: water, saline, buffers, inert, nontoxic solids (e.g., mannitol, talc). Compositions comprising such carriers are formulated by well-known conventional methods. Depending on the intended mode of administration and the intended use, the compositions may be in the form of solid, semi-solid, or liquid dosage forms, such, for example, as powders, granules, crystals, liquids, suspensions, liposomes, pastes, creams, salves, etc., and may be in unit-dosage forms suitable for administration of relatively precise dosages. Preferably, the pharmaceutical composition according to the invention is administered in therapeutically effective amount.

“A therapeutically effective amount” as used herein refers to an amount necessary to promote differentiation of pancreatic progenitor cells into cells of the pancreatic endocrine lineage. A therapeutically effective amount differs according to the administration route, excipient usage and co-usage of other active agents.

The present invention also relates to a method of treatment of a subject having a pancreatic disorder, preferentially having pancreatic disorder, more preferably having diabetes of type 1 or 2, by administering to said subject at least one BET inhibitor or a pharmaceutical composition comprising at least one BET inhibitor according to the invention.

The present invention further relates to a measure of the efficiency of a BET inhibitor treatment in a subject having a pancreatic disorder, preferentially having pancreatic disorder, more preferably having diabetes of type 1 or 2, comprising the steps of:
a. evaluating control glucose homeostasis from biological samples of said subject;
b. administering the BET inhibitors or a pharmaceutical composition of BET inhibitors to said subject according to the invention;
c. evaluating control glucose homeostasis, after a defined period of time, from biological samples of said subject.

The term “biological sample” refers to all materials that are produced by biological organisms or can be isolated from them; in particular, it refers to materials allowing the determination of the glucose level in a subject. The term “biological sample” includes untreated or pretreated samples, e.g. plasma, body fluids, preferentially blood.

The terms “defined period of time” refer herein to the time required for the BET inhibitor to induce its therapeutic effect in the subject. The “defined period of time” can be from several hours to several days, preferentially from 1 to 2 days.

### DESCRIPTION OF THE FIGURE

FIG. 1: BET inhibitors induce a major increase of neurogenin 3 expression. A, Mouse pancreatic buds were cultured in presence of either DMSO, 0.5μM I-BET 151 or 0.1μM (+)-JQ1. After 1, 3, 5 or 7 days, total RNA was extracted. Relative expression of Ngn3 was measured by RT-qPCR normalized with Cyclophylin A expression. Values represent the average of three independent experiments with standard deviation error bars. B and C, NGN3 expression was analyzed by immunohistochemistry on paraffin embedded mouse pancreatic buds that were cultured for five days in presence of DMSO, 0.5μM I-BET 151 or 0.1μM (+)-JQ1. B, Representative image of NGN3 staining. Scale bar measures 100μm. C, Total NGN3 positive nuclei per rudiment were visually counted. D. Mouse pancreatic buds were culture in presence of either DMSO, 0.5 μM I-BET 151 or 0.1μM (+)-JQ1 during 5 days and then cultured for 9 additional days with complete medium only. Ins1, Ins 2 and MafA expression was measured by RT-qPCR normalized with Cyclophylin A expression. Values represent the average of three independent experiments with standard deviation error bars. *P≤0.05 **P≤0.01 ***P≤0.001.

FIG. 2: Relative Ngn3 mRNA expression in iPSC-derived endocrine progenitors treated with BET inhibitors. A Overview of the protocol for directed differentiation of pluripotent stem cells towards endocrine progenitors. Schematic overview outlining the five stages of the protocol for differentiating pluripotent stem cells towards pancreatic endocrine progenitors.

The timing of the individual stages (in days) and proteins and chemicals used for each step of the protocol are listed under each stage. iPSC-derived pancreatic endoderm was differentiated towards endocrine progenitors for three days in the presence of varying (+)-JQ1 B or IBET-151 C concentrations. Following the differentiation, mRNA was harvested from the cells and Ngn3 mRNA expression was analysed by qPCR. Graphs shows mean±SD of one experiment with two technical replicates.

FIG. 3: Viability and cell number following BET inhibitor treatment. iPSC-derived pancreatic endoderm was differentiated towards endocrine progenitors for three days in the presence of varying (+)-JQ1 A or IBET-151 B. Following the differentiation, cells were harvested from wells and cell number and viability was assessed by a Nucleocounter image cytometer. (●) shows the percentage of live cells (left Y-axis of graphs). (▪) shows cell number adjusted to growth area (right y-axis of graphs). Graphs shows mean±SD of one experiment with two technical replicates.

FIG. 4: Neurogenin 3 protein expression in iPSC-derived endocrine progenitors treated with BET inhibitors. iPSC-derived pancreatic endoderm was differentiated towards endocrine progenitors for three days in the presence of 500 nM (+)-JQ1, 2000 nM IBET-151 or vehicle control (DMSO). Following the differentiation, cells were fixed and processed for immunohistochemistry. Cells were stained for Neurogenin 3 (NGN3) using a polyclonal

Neurogenin 3 antibody and the nuclei of all cells were visualized using DAPI. Scalebar in images are 500uM in A and 200 uM in B. Representative images of three independent experiments are shown. The same exposure time was used for both control and BET inhibitor treated wells.

FIG. 5: Quantification of Neurogenin 3 protein expressing cells following BET inhibitor treatment. iPSC-derived pancreatic endoderm was differentiated towards endocrine progenitors for three days in the presence of 400 nM (+)-JQ1, 2000 nM IBET-151 or vehicle control (DMSO). Cells were subsequently harvested and percentage of cells expressing Neurogenin 3 was analyzed by flow cytometry. A Representative dot plots of cells treated with DMSO, (+)-JQ1 or IBET-151 for three days. X-axis shows Neurogenin 3 signal (Ngn3), Y-axis shows side scatter signal (SSC). Gates were set according to isotype controls. Numbers in dot plots shows percentage of cells within the gate. B Percentage of Neurogenin 3 (Ngn3) positive cells analyzed across three independent differentiation experiments, using a different iPSC line for each experiment. Boxes in graph show min to max with line at the mean. Individual biological replicates are shown as dots on the graph. C A human ESC-line genetically modified to express green fluorescence protein (GFP) under the control of Neurogenin 3 was differentiated to pancreatic endoderm and subsequently differentiated to the endocrine progenitor stage for three days in the presence of 400 nM (+)-JQ1, 2000 nM IBET-151 or vehicle control (DMSO). X-axis shows the GFP signal, Y-axis shows side scatter signal (SSC).

### EXAMPLES

### Example 1

### Dissection and Culture

Mouse pancreatic buds were dissected from E11.5 C57Bl6/J embryos and placed at the air/medium interface on 0.45μm filters in culture dishes containing RPMI medium supplemented with 10% fetal calf serum, 1% penicillin-streptomycin, 1% non-essential amino acids and 10mM HEPES. Pancreatic buds were cultured at 37° C. with 5% CO₂.

### Treatments

IBET-151 was obtained from Sigma-Aldrich and (+)/-JQ1 from Abcam. Stock solutions were prepared in DMSO. Inhibitors or DMSO (0.1% final concentration) were added to the culture medium which was changed daily.

### Real Time PCR Analyses

Total RNA from three or more pancreatic buds was extracted using Qiagen RNA extraction microkit and 250ng were reverse transcribed using maxima first strand cDNA synthesis kit from Thermo Fisher. Real-time PCR analysis of Ngn3, MafA and Cyclophylin A were performed in 1× Sybr Green Powermix in QuantStudio 3 Applied Biosystem system. Real-time PCR analysis of Ins1 and Ins2 were performed in 1× TaqMan Gene Expression Mastermix in a QuantStudio 3 Applied Biosystem system. Relative expression of Ngn3, MafA and Cyclophylin A were calculated using the comparative method of relative quantification (2^{-ΔΔCT}) normalized to cyclophilin A expression. Values represent the average of three experiments with standard deviation error bars. Statistical analysis was performed using unpaired Student t test.

### Immunohistochemistry and Quantification

Pancreatic buds were fixed in 3.7% formaldehyde, pre-embedded in agarose gel (4% of type VII low gelling temperature agarose (Sigma-Aldrich)) and embedded in paraffin. Sections (4 μm thick) were collected and processed for 3,3' diaminobenzidine immunohistological staining of NGN3, as previously described (Attali et al., 2007).

Photographs representative of a whole pancreas were taken using a transmitted light microscope (Leitz DMRB, Leica) and digitized using a Hammamatsu cooled 3CCCD camera. Total number of NGN3 positive nuclei per rudiment were then manually counted. Values represent the average of three experiments with standard deviation error bars. Statistical analysis was performed using unpaired Student t test.

### Results

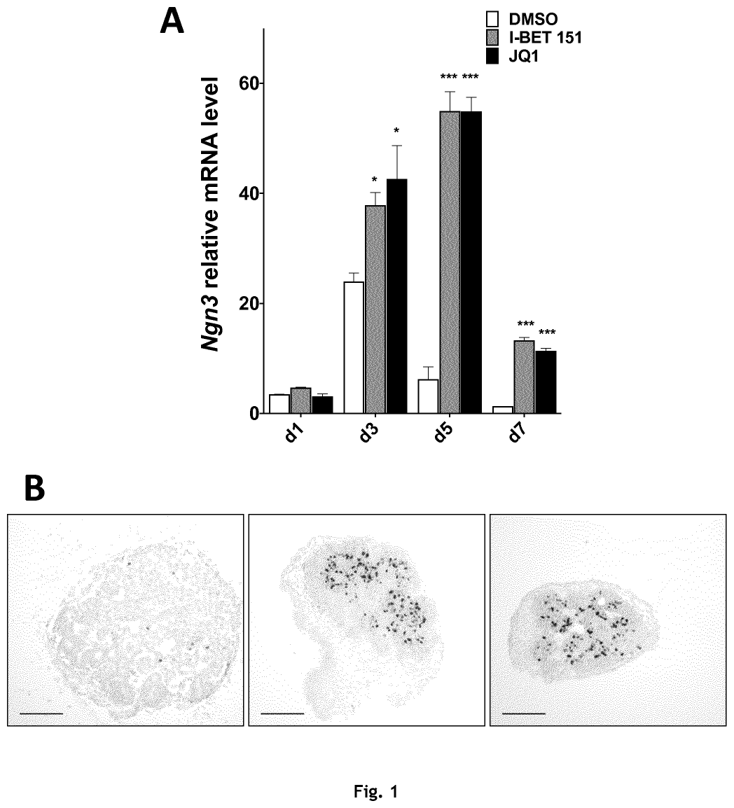

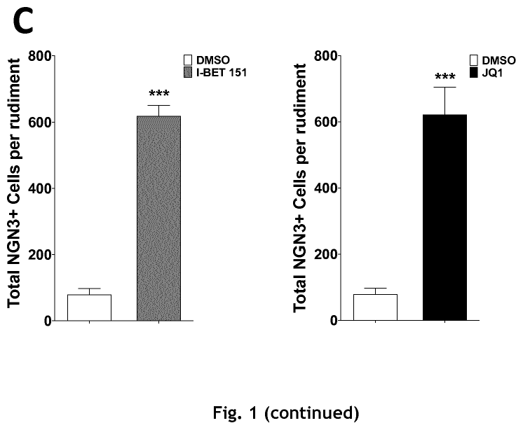

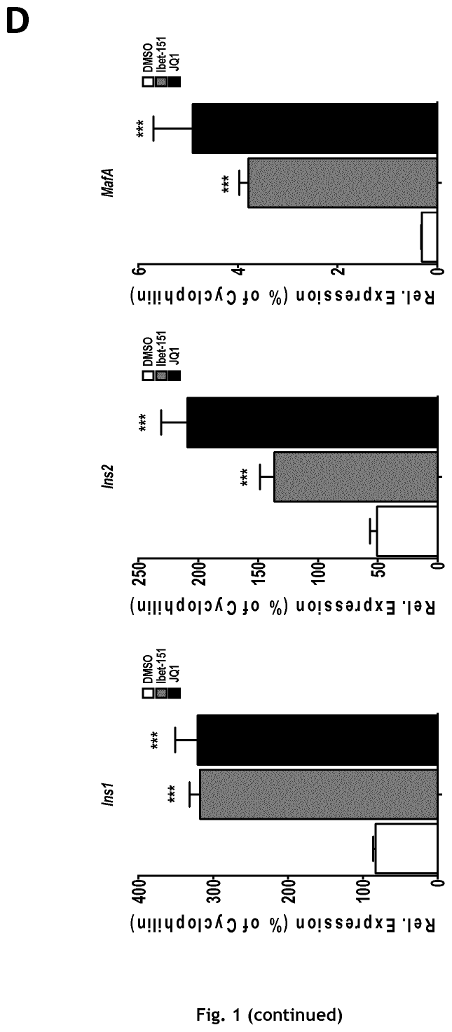

We studied the effects of IBET-151 and JQ1 on mouse embryonic pancreatic buds, which were cultured for 1, 3, 5 or 7 days in presence of each inhibitor (0.5 μM of IBET-151 or 0.1 μM of (+)-JQ1). Here we show that Ngn3 relative mRNA levels are strongly increased after 3 days of treatment, and remain increased after 7 days (FIG. 1A). These results were further confirmed by quantitative immunohistochemistry. They indicate that the number of NGN3 positive nuclei is increased by BET inhibitors treatment (FIGS. 1B and 1C). To evaluate the potential ability of the increased NGN3 positive population to undergo endocrine differentiation into mature B cells, E11.5 pancreatic buds were cultured for 5 days with IBET-151 or (+)-JQ1. Buds were then washed with fresh culture medium devoid of inhibitors and kept in culture for 9 additional days. Interestingly, Ins1 and Ins2 expressions were increased by 3 fold when pancreatic buds had previously been exposed to IBET-151 or (+)-JQ1 (FIG. 1D). Moreover, the expression of MafA, another marker of mature B cells, was increased by 10 folds (FIG. 1D). Altogether, these results indicate that IBET-151 and (+)-JQ1 stimulate multipotent progenitors toward endocrine differentiation, and that the increased NGN3 positive population can ultimately lead to more insulin and MafA expression.

These results hence suggest that these two inhibitors stimulate multipotent progenitors toward endocrine differentiation.

### Example 2

### Directed Differentiation of Pluripotent Stem Cells to Pancreatic Endocrine Progenitors.

Human induced pluripotent stem cells (iPSC) and human embryonic stem cells (ESC) were cultured on standard tissue culture plastic ware coated with hESC-qualified matrigel in mTeSR1^{™}medium. Cells were passaged every three to four days as single cells using TrypLe Select. Rock inhibitor (5 μM) was included at the first day of passaging. Three different iPSC lines derived from a total of two individual donors and one ESC line were applied.

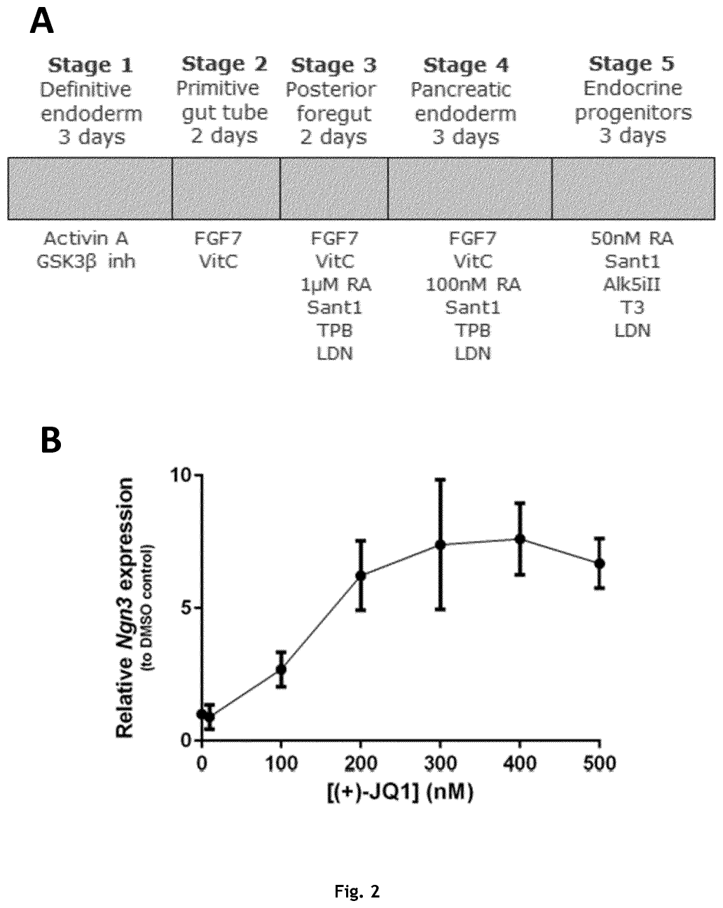

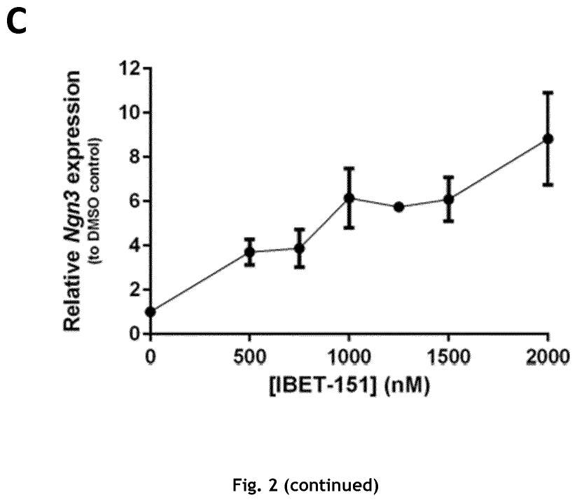

For differentiation, cells were seeded as single cells in mTeSR1 with 5 μM Rock inhibitor into tissue culture plates (Corning CellBind) at densities ranging between 300-400.000 cells/cm². Cells were cultured for 24 h at 37° C., 5% CO₂. Following incubation, medium was aspirated and the cells were washed once in PBS before adding the differentiation medium. Differentiation was carried out essentially as described in Rezania et al. (Rezania et al., 2014b). The differentiation protocol is outlined in FIG. 2A. Medium was replenished daily according the list below:
Stage 1—Definitive endoderm (3 days):
Day 1:MCDB131-1 medium* with 100 ng/ml Activin and 3 μM CHIR
Day 2:MCDB131-1 medium* with 100 ng/ml Activin and 0.3 μM CHIR
Day 3:MCDB131-1 medium* with 100 ng/ml Activin
Stage 2—primitive gut tube (2 days):
Day 4-5:MCDB131-1 medium* with 0.25 mM Ascorbic acid and 50 ng/ml KGF
Stage 3—Posterior foregut (2 days):
Day 6-7:MCDB131-2 medium** with 0.25 mM Ascorbic acid, 50 ng/ml KGF, 1 μM Retinoic acid, 0.25 μM Sant-1, 100 nM LDN and 200 nM TPB.
Stage 4—Pancreatic endoderm (3 days):
Day 8-10 MCDB131-2 medium** with 0.25 mM Ascorbic acid, 2ng/ml KGF, 0.1 μM Retinoic acid, 0.25 μM Sant-1, 200 nM LDN and 100 nM TPB.
Stage 5—Endocrine progenitors (3 days):
Day 8-10:MCDB131-3 medium*** with 0.05 μM Retinoic acid, 0.25 μM Sant-1, 100 nM LDN, 10 μM Alk5ill, 10μg/ml heparin and 1 μM T3. At this stage, varying concentrations of BET inhibitors (IBET-151 or (+)-JQ1) or concentration matched vehicle control (DMSO) were included in the differentiation medium.

### Medium Details:

| *MCDB131-1 | **MCDB131-2 | ***MCDB131 medium |
|---|---|---|
| MCDB131 medium | MCDB131 medium | MCDB131 medium |
| 0.1% Pen/Strep | 0.1% Pen/Strep | 0.1% Pen/Strep |
| 1.5 g/L NaHCO3 | 2.5 g/L NaHCO3 | 1.5 g/L NaHCO3 |
| 1 x Glutamax | 1 x Glutamax | 1 x Glutamax |
| 10 mM Glucose | 10 mM Glucose | 20 mM Glucose |
| final | | |
| 0.5% BSA | 2% BSA | 2% BSA |
| | 0.25 mM Ascorbic Acid | 1:200 ITS-X |
| | 22 mg/ml AA solution | |
| | 1:200 ITS-X | 10 μM Zinc |
| | | sulfate |

This protocol consistently yields >90% Sox17-positive cells with <5% Oct4 cells at the end of stage 1 (definitive endoderm) and between 40-70% PDX1/NKX6-1 co-positive cells at the end of stage 4 (pancreatic endoderm) depending on cell lines used (data not shown).

Viability of the cells and total number of cells was analyzed using a Nucleocounter NC3000 Cell analyzer (Chemometec).

### Flow Cytometry Analysis

Differentiation efficiency was analyzed by flow cytometry essentially as described in van de Bunt et al., 2016. Briefly, cells were harvested from wells by TrypLe select and subsequently quenched for 20 min in 4% formalin on ice. Fixed cells were washed once in PBS and then permeabilized for 30 min on ice in PBS containing 5% donkey serum and 0.2% Triton-X100. Following permeabilization, cells were stained with primary antibodies diluted in PBS+5% donkey serum+0.1% Triton-X100 for 30 min at room temperature (directly conjugated antibodies) or overnight at 4°C. (unconjugated antibodies). Cells were washed once in PBS with 1% bovine serum albumin. Unconjugated antibodies were detected with fluorophore conjugated secondary antibodies. The following antibodies were used:

| | | | Catalog | Final |
|---|---|---|---|---|
| Antigen | Conjugate | Vendor | no. | dilution |
| Sox17 | Alexa488 | BD Pharmingen | 562205 | 1:40 |
| PDX1 | Alexa488 | BD Pharmingen | 562274 | 1:40 |
| NKX6.1 | Alexa647 | BD Pharmingen | 563338 | 1:40 |
| Oct4 | Alexa647 | BD Pharmingen | 560329 | 1:10 |
| Neurogenin 3 | None | R&D systems | AF3444 | 1:200 |

RNA Isolation, cDNA Synthesis and Quantitative PCR (qPCR).

RNA was isolated from cells using NucleoSpin RNA/protein isolation kit (Macherey-Nagel). RNA was quantified using a nanodrop and 500-1000 μg RNA pr. sample was converted to cDNA using iScript reverse transcription kit (Bio-Rad). Gene expression was evaluated using Tagman gene expression assay for Neurogenin 3(Hs01875204, Applied Biosystems).

Neurogenin 3 transcripts were normalized to the average expression of two housekeeping genes (ACTB, Hs01060665_g1 and HPRT1, Hs99999909_m1, both from Applied Biosystems). Relative expression was calculated using the ΔΔCt method (FIGS. 2B and 2C).

### Immunohistochemistry Analysis

Immunohistochemistry analysis was performed as described in van de Bunt et al., 2016. Briefly, cells were fixed directly in tissue culture plates and subsequently permeabilized in PBS+05% Triton-X100 for 10 minutes and blocked in a tris-buffer containing 0.5% Tyramide Signal Amplification (TSA) immunohistochemistry kit blocking reagent for 30 min at room temperature. Cells were incubated with an anti-Neurogenin 3 antibody diluted in PBS+0.1% Triton-X100 (R&D systems, AF3444) overnight at 4° C. Cells were washed thrice in PBS and specific binding of the Neurogenin 3 antibody was revealed using a fluorescence coupled secondary antibody. Nuclei of all cells was revealed using 4′,6-diamidino-2-phenylindole.

### Results and Discussion

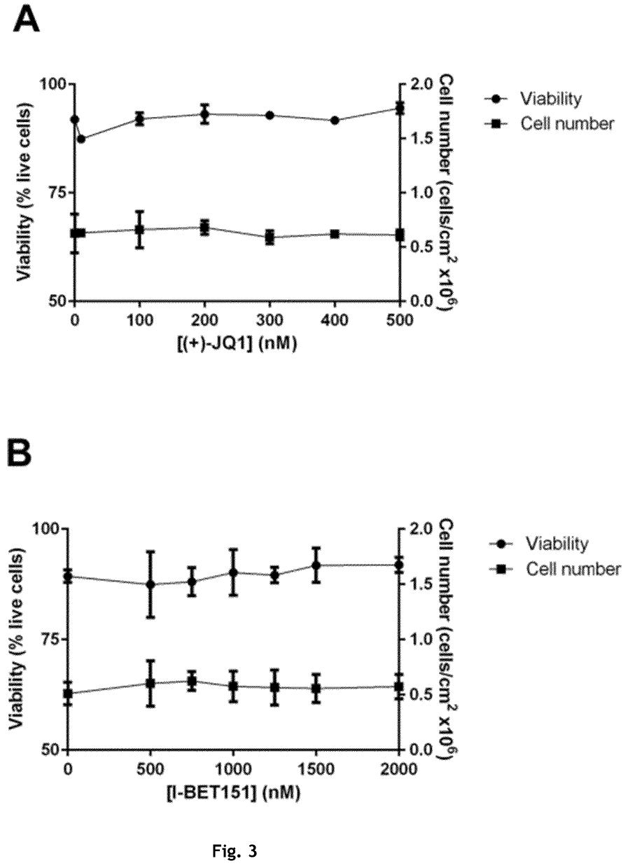

The effect of the BET inhibitors on Neurogenin 3 induction was tested in the context of human pluripotent stem cell differentiation. hiPSC were differentiated towards the pancreatic lineage using directed differentiation as described in the materials and methods (FIG. 2A). The hiPSC-derived pancreatic progenitors (also termed pancreatic endoderm) were differentiated towards the pancreatic endocrine lineage for three days in the presence of six different concentrations of either of the two BET inhibitors. Following the differentiation, induction of Neurogenin 3 expression was assessed by qPCR. A clear dose-dependent increase of Neurogenin 3 mRNA expression was observed for both of the BET inhibitors, with the maximum expression achieved at 300-400 nM JQ1 or 2000 nM IBET151. Potential toxic effect of the BET inhibitors on the differentiated cells was also evaluated. In the same experiment as described above, cells were harvested following the three day treatment and viability and cell number was determined using a Nucleocounter. No obvious difference in both cell number and viability was observed across all tested concentrations of the BET inhibitors compared to the controls (concentration matched DMSO) (FIG. 3A, B). These results suggest that the BET inhibitors can induce expression of Neurogenin 3 mRNA in hiPSC differentiated towards pancreatic progenitors.

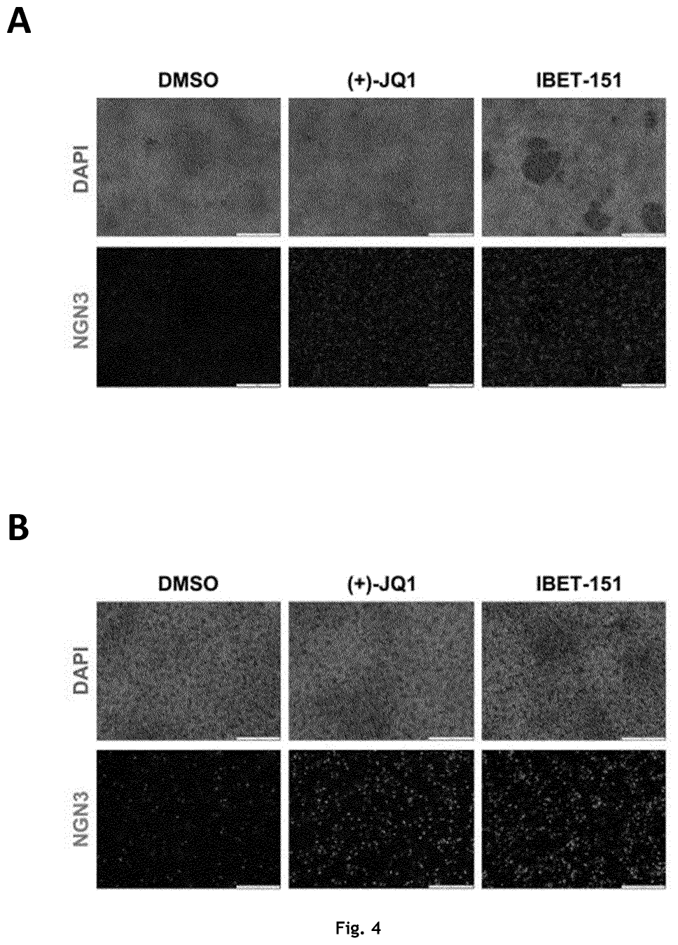

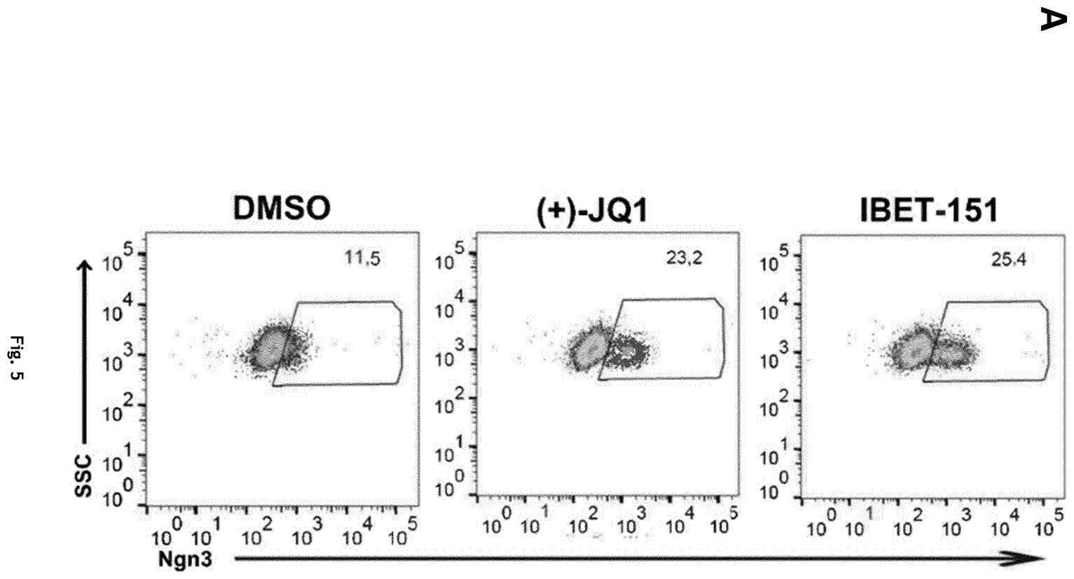

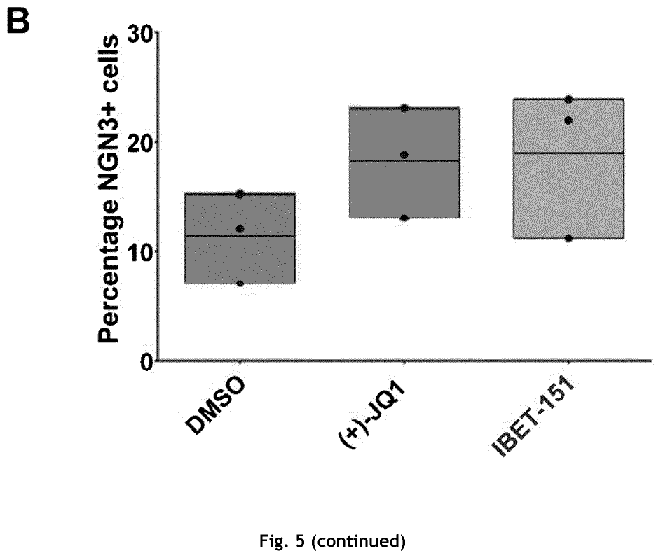

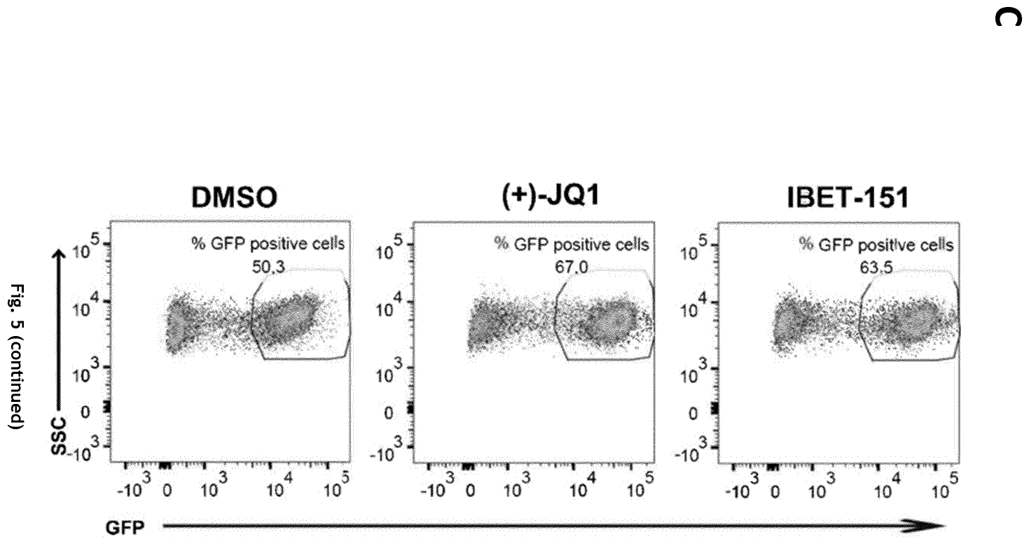

To determine whether the induction of Neurogenin 3 mRNA by the BET inhibitors also resulted in increased expression of Neurogenin 3 protein in the differentiated hiPSC, cells were fixed following BET inhibitor treatment and Neurogenin 3 protein expression was evaluated by immunofluorescence microscopy and flow cytometry. FIGS. 4A and B shows representative images of hiPSC-derived endocrine progenitor cells treated for three days with JQ1, IBET 151 or DMSO as control. A clear increase in the number of cells positive for Neurogenin 3 protein is detected. The staining intensity of Neurogenin 3 in individual cells appears stronger in the cells treated with the BET inhibitors compared to the DMSO control, suggesting that there is more Neurogenin 3 protein present in individual cells (FIG. 4A, B). The percentage of cells expressing Neurogenin 3 protein following treatment with the BET inhibitors was determined using flow cytometry. Across three biological experiments, the number of cells expressing Neurogenin 3 protein was approximately 1.6 fold higher when treated with one of the BET inhibitors compared to the control treated cells (FIGS. 5A and B). In order to test the effect of the BET inhibitors on a hESC we applied a genetically modified hESC line that express green fluorescence protein (GFP) under the control of the Neurogenin 3 promoter. When this hESC line was differentiated to pancreatic progenitors and further towards the endocrine lineage for three days, more cells expressing GFP was observed when the cells were differentiated in the presence of either of the two BET inhibitors compared to the control. Together, these results demonstrate the ability of JQ1 and IBET 151 to induce the expression of Neurogenin 3 mRNA and protein during the differentiation of human pluripotent stem cells (both hiPSC and hESC) towards the pancreatic endocrine lineage.

### Conclusion/Summary

JQ1 and IBET 151 dose-dependently induces Neurogenin 3 mRNA expression in hiPSC-derived pancreatic progenitors differentiated towards the endocrine lineage.
JQ1 and IBET 151 induce Neurogenin 3 protein expression in pancreatic endocrine progenitors.
No obvious toxicity or influence on cell number was detected on the differentiated human pluripotent stem cells by either of the BET inhibitors.
The induction of Neurogenin 3 expression during the pancreatic endocrine differentiation is applicable to both hESC and hiPSC.

### REFERENCES:

Attali, M., Stetsyuk, V., Basmaciogullari, A., Aiello, V., Zanta-Boussif, M. A., Duvillie, B., and Scharfmann, R. (2007). Control of beta-cell differentiation by the pancreatic mesenchyme. Diabetes 56, 1248-1258.
Avolio, F., Pfeifer, A., Courtney, M., Gjernes, E., Ben-Othman, N., Vieira, A., Druelle, N., Faurite, B., and Collombat, P. (2013). From pancreas morphogenesis to beta-cell regeneration. Current topics in developmental biology 106, 217-238.
Bhushan, A., Itoh, N., Kato, S., Thiery, J., Czernichow, P., Bellusci, S., and Scharfmann, R. (2001). Fgf10 is essential for maintaining the proliferative capacity of epithelial progenitor cells during early pancreatic organogenesis. Development 128, 5109-5117.
Chen, S., Borowiak, M., Fox, J. L., Maehr, R., Osafune, K., Davidow, L., Lam, K., Peng, L. F., Schreiber, S. L., Rubin, L. L., et al. (2009). A small molecule that directs differentiation of human ESCs into the pancreatic lineage. Nat Chem Biol 5, 258-265.
D'Amour, K. A., Bang, A. G., Eliazer, S., Kelly, O. G., Agulnick, A. D., Smart, N. G., Moorman, M. A., Kroon, E., Carpenter, M. K., and Baetge, E. E. (2006). Production of pancreatic hormone-expressing endocrine cells from human embryonic stem cells. Nat Biotechnol 24, 1392-1401.
Elghazi, L., Cras-Meneur, C., Czernichow, P., and Scharfmann, R. (2002). Role for FGFR2IIIb-mediated signals in controlling pancreatic endocrine progenitor cell proliferation. Proc Natl Acad Sci USA 99, 3884-3889.
Gittes, G. K. (2009). Developmental biology of the pancreas: a comprehensive review. Dev Biol 326, 4-35.
Haumaitre, C., Lenoir, O., and Scharfmann, R. (2008). Histone deacetylase inhibitors modify pancreatic cell fate determination and amplify endocrine progenitors. Mol Cell Biol 28, 6373-6383.
Jennings, R. E., Berry, A. A., Strutt, J. P., Gerrard, D. T., and Hanley, N. A. (2015). Human pancreas development. Development 142, 3126-3137.
Kroon, E., Martinson, L. A., Kadoya, K., Bang, A. G., Kelly, O. G., Eliazer, S., Young, H., Richardson, M., Smart, N. G., Cunningham, J., et al. (2008). Pancreatic endoderm derived from human embryonic stem cells generates glucose-responsive insulin-secreting cells in vivo. Nat Biotechnol 26, 443-452.
Miralles, F., Czernichow, P., Ozaki, K., Itoh, N., and Scharfmann, R. (1999). Signaling through fibroblast growth factor receptor 2b plays a key role in the development of the exocrine pancreas. Proc Natl Acad Sci USA 96, 6267-6272.
Pagliuca, F. W., Millman, J. R., Gurtler, M., Segel, M., Van Dervort, A., Ryu, J. H., Peterson, Q. P., Greiner, D., and Melton, D. A. (2014). Generation of functional human pancreatic beta cells in vitro. Cell 159, 428-439.
Pan, F. C., and Wright, C. (2011). Pancreas organogenesis: from bud to plexus to gland. Dev Dyn 240, 530-565.
Rezania, A., Bruin, J. E., Arora, P., Rubin, A., Batushansky, I., Asadi, A., O'Dwyer, S., Quiskamp, N., Mojibian, M., Albrecht, T., et al. (2014a). Reversal of diabetes with insulin-producing cells derived in vitro from human pluripotent stem cells. Nature biotechnology 32, 1121-1133.
Van de Bunt M, Lako M, Barrett A et al. Insights into islet development and biology through characterization of a human iPSC-derived endocrine pancreas model. Islets 2016; 8: 83-95.
Ye, F., Duvillie, B., and Scharfmann, R. (2005). Fibroblast growth factors 7 and 10 are expressed in the human embryonic pancreatic mesenchyme and promote the proliferation of embryonic pancreatic epithelial cells. Diabetologia 48, 277-281.

1. In vitro method for obtaining cells of the pancreatic endocrine lineage, comprising a step of culturing pancreatic progenitor cells, wherein said pancreatic progenitor cells are in a cell culture medium comprising at least one BET inhibitor, and wherein said pancreatic progenitor cells are obtained by differentiation of stem cells obtained by techniques that do not involve the destruction of a human embryo.

2. The in vitro method according to claim 1, wherein the at least BET inhibitor is comprised in a concentration from 10 nM to 10 μM.

3. The in vitro method according to claim 1 or 2, wherein the at least BET inhibitor is targeting BD1 and/or BD2.

4. The in vitro method according to claim 3, wherein the at least BET inhibitor targeting BD1 and/or BD2 is selected in the group comprising BET 151, JQ1, BET762, OXT-015, TEN-010, CPI-203, CPI 0610, LY29002 and RVX8, preferentially BET 151 and JQ1.

5. The in vitro method according to claim 1, wherein said pancreatic progenitor cells are obtained from embryonic stem cells, perinatal stem cell, somatic stem cells, and bioengineered stem cells, preferably said stem cells are hESC or iPSC, in particular hi PSC.

6. The in vitro method according to any of the previous claims wherein said pancreatic progenitor cells are cultured in said cell culture medium for at least 8 hours, preferably for at least 24 hours, more preferably for 48 hours, even more preferably 72 hours.

7. A cell of the pancreatic endocrine lineage obtainable by a method according to any of the previous claims.

8. A cell of the pancreatic endocrine lineage according to claim 6, for use as a medicament

9. A cell of the pancreatic endocrine lineage according to claim 6 or 7, for its use as a medicament for treating or preventing a pancreatic disorder, preferably chosen in the list consisting of pancreatitis, such as acute pancreatitis and chronic pancreatitis, diabetes mellitus, exocrine pancreatic insufficiency (EPI), cystic fibrosis (also known as mucoviscidosis), congenital malformations, such as pancreas divisum and annular pancreas, neoplasms (such as serous cystadenoma of the pancreas, solid pseudopapillary neoplasm or Zollinger-Ellison syndrome), and Hemosuccus pancreaticus.

10. A cell of the pancreatic endocrine lineage for use according to claim 8, wherein said pancreatic disorder is diabetes mellitus, preferably type I or type II diabetes.

11. Use of a cell of the pancreatic endocrine lineage obtainable by a method of any one of claims 1 to 5 for the in vitro production of insulin.

12. Use of a cell of the pancreatic endocrine lineage obtainable by a method of any one of claims 1 to 5 for the in vitro identification of compounds capable of modulating insulin production.

13. At least one BET inhibitor for use for treating or preventing a pancreatic disorder, preferably chosen in the list consisting of pancreatitis, such as acute pancreatitis and chronic pancreatitis, diabetes mellitus, exocrine pancreatic insufficiency (EPI), cystic fibrosis (also known as mucoviscidosis), congenital malformations, such as pancreas divisum and annular pancreas, neoplasms (such as serous cystadenoma of the pancreas, solid pseudopapillary neoplasm or Zollinger-Ellison syndrome), and Hemosuccus pancreaticus.

14. At least one BET inhibitor for use according to claim 13, wherein said pancreatic disorder is diabetes mellitus, preferably type I or type II diabetes.

15. Pharmaceutical composition comprising at least one BET inhibitor according to claim 13 or 14 and a pharmaceutically acceptable carrier.
